(19) United States
(12) Patent Application Publication
(10) Pub. No.: US 2014/0235840 A1
(43) Pub. Date: Aug. 21, 2014

(54) NEW SYNTHESIS OF FUCOSE

(71) Applicant: GLYCOM A/S, Kongens Lyngby (DK)

(72) Inventors: Nikolay Khanzhin, Humlebaek (DK); Julien Boutet, La Plaine sur Mer (FR); Filippo Bonaccorsi, Livorno (IT); Gyula Dekany, Queensland (AU)

(21) Appl. No.: 14/348,432

(22) PCT Filed: Sep. 28, 2012
(86) PCT No.: PCT/IB2012/055211

§ 371 Date: Mar. 28, 2014

(30) Foreign Application Priority Data
Sep. 30, 2011 (EP) 11183618.5

(51) Int. Cl. C07H 1/00; C07H 3/02; C07H 9/04

(52) U.S. Cl. CPC C07H 1/00; C07H 9/04; C07H 3/02
USPC 536/18.1; 536/124

(57) ABSTRACT

A process for converting D-glucose into L-fucose, where a first aspect of the disclosure relates to a method of making a compound of formula (1) wherein R is independently H, alkyl or phenyl or, preferably, wherein the two germinal R groups together with the carbon atom to which they are attached form a C3-s cycloalkylidene group, including the step of treating a compound of formula (2) wherein R is defined above and R₁ is a sulphonate leaving group, with a reducing complex metal hydride and, preferably, a base to form the compound of formula (1); a compound of formula (13).

### FIELD OF THE INVENTION

L-Monosaccharides or L-sugars, especially L-hexoses, are scarce in nature. Nevertheless, some L-hexoses are key building blocks in biologically important oligosaccharides, glycopeptides and other glycoside type derivatives among which L-fucose (6-deoxy-L-galactose) and L-rhamnose (6-deoxy-L-mannose) are best known.

Owing to their biological and medicinal properties and their scarcity in nature, chemists have developed synthetic processes or pathways for making L-sugars from abundant and cheap D-sugars. Generally, these synthetic pathways have included the extensive use of selective protective group manipulations and regio- and/or stereoselective functional group transformations such as S_{N}2-type inversions (epimerization), oxidation-reduction sequences, β-eliminations, additions to double bonds including C═O and/or C═C double bonds, and deoxygenations. These synthetic pathways have commonly included several steps, in which process intermediates have often needed to be isolated from reaction mixtures and purified prior to the next process steps.

For example, D-glucose has been converted into 6-deoxy-1,2-O-isopropylidene-β-L-talofuranose (compound F), a compound serving as precursor for modified nucleoside analogs (Zsoldos-Mády et al. Monatsh. Chem. 117, 1325 (1986), Hiebl et al. ibid. 121, 691 (1990)) and for chiral diphosphite ligands for asymmetric catalytic reactions (Diéguez et al. Chem. Eur. J. 7, 3086 (2001)). See the three pathways in Scheme 1 below. All three pathways have a common route from D-glucose to 3-O-acetyl-1,2-O-isopropylidene-α-D-allofuranose (compound A) in five steps. Compound A was then converted into the epoxide of formula E1 or E2 in four steps involving the introduction of a sulphonate leaving group in position 5 via regio- and chemoselective protective group manipulations, and the epoxides were then treated with LiAlH₄ to give compound F. All the three pathways have involved as many as ten elementary functional group transformations which have made each process cumbersome, inefficient and hence unattractive for industrial application.

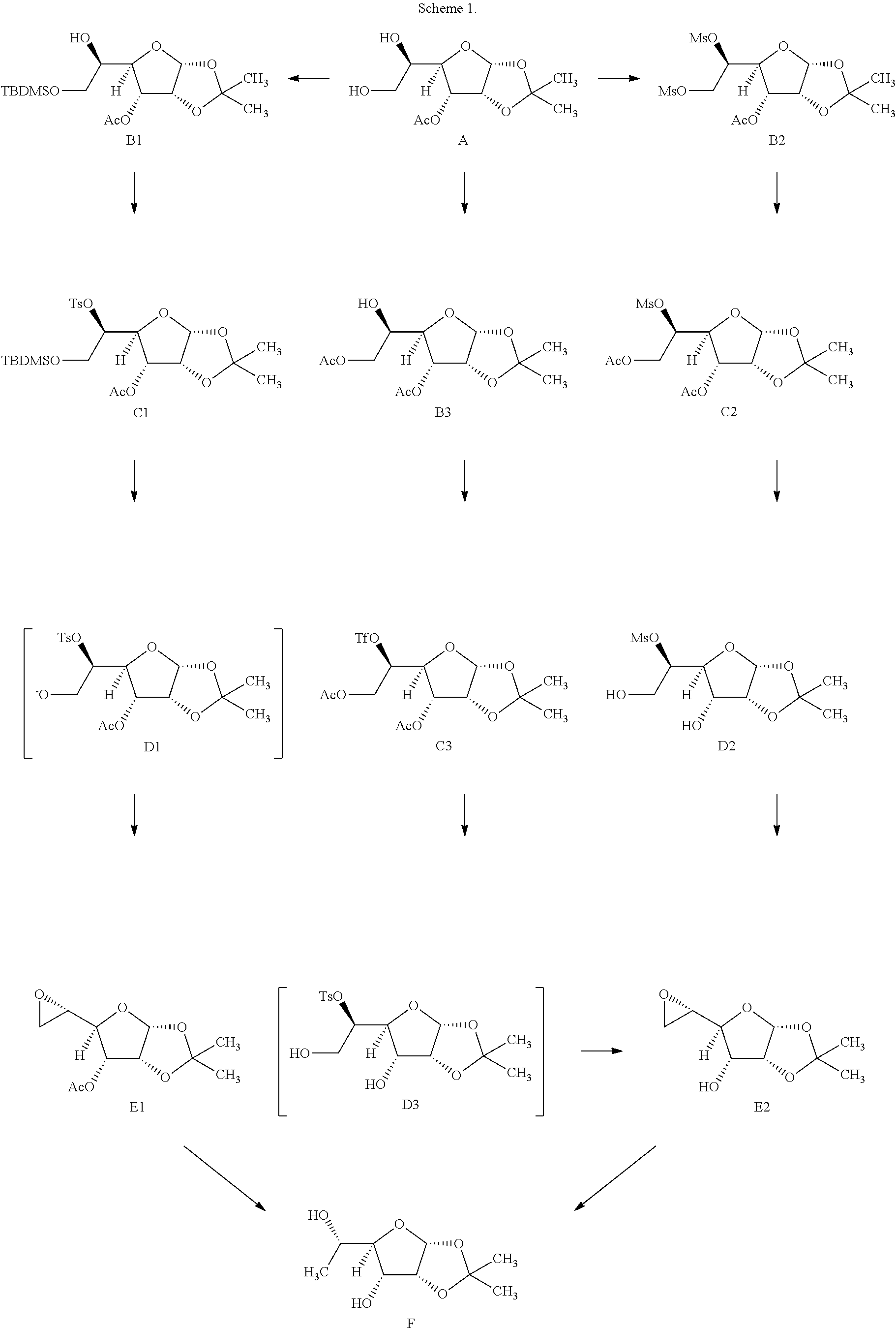

Although numerous synthetic processes have been developed to convert readily available cheap D-sugars into L-sugars, there has been a need for processes which take less time, require fewer reagents/solvents and/or provide better yields.

### SUMMARY OF THE INVENTION

The present invention provides a process for converting D-glucose into L-fucose. In this process, fewer steps are required and the need for OH-protection is reduced compared with prior processes. As a result, the process can readily be carried out on a large scale, for efficient commercial production of L-fucose.

A first aspect of this invention relates to a method of making a compound of formula 1

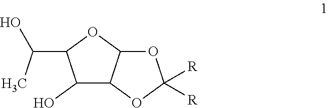

wherein R is independently H, alkyl or phenyl or, preferably,
wherein the two geminal R groups together with the carbon atom to which they are attached form a C₃₋₈ cycloalkylidene group,
   comprising the step of treating a compound of formula 2

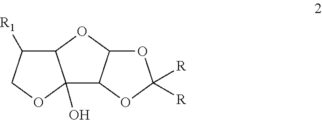

wherein R is as defined above and R₁ is a sulphonate leaving group, with a reducing complex metal hydride and optionally a base.

A second aspect of the invention relates to compounds of formula 13

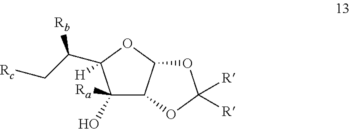

wherein the moiety

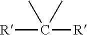

is a highly lipophilic protecting group and wherein either: Rₐ and R_{c} together form an oxygen bridge when
R_{b} is OH or a sulphonate leaving group; or Rₐ is H and R_{c} is OH
when R_{b} is a sulphonate leaving group,
   and formula 14

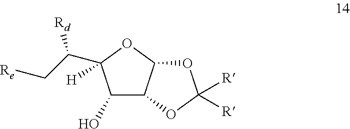

wherein the moiety

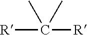

is a highly lipophilic protecting group, and wherein either: R_{d} is OH and Rₑ is H; or R_{d} and Rₑ together form an oxygen bridge.

A third aspect of the invention relates to the use of the compounds of the second aspect of the invention in the synthesis of 6-deoxy-L-talose or L-fucose.

### DETAILED DESCRIPTION OF THE INVENTION

In this invention, the term “highly lipophilic protecting group” preferably means a protecting group, such as a longer alkyl chain ketal group or a cyclic ketal group, for a compound that is a process intermediate. Such a protecting group makes the intermediate more lipophilic and thus more soluble in organic solvents. In preferred “highly lipophilic protecting groups”, the moiety

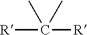

is a hydrocarbon group of at least 5 carbon atoms, preferably wherein R′ is a C₂₋₆ alkyl or phenyl or wherein the two geminal R′ groups together with the carbon atom to which they are attached form a C₅₋₈ cycloalkylidene, particularly preferably wherein the two R′ groups together with the carbon atom to which they are attached form a cyclohexylidene.

Herein, the term “sulphonate leaving group” means a conventional sulphonate ester which can be displaced by a nucleophile in nucleophilic substitution reactions. More specifically, a sulphonate leaving group can be represented by the formula —OSO₂—R*, wherein R* means an alkyl group optionally substituted with one or more halogen atoms, preferably fluoro, an optionally substituted homoaromatic group selected from phenyl and naphthyl, or an optionally substituted 5-10 membered mono- or bi-cyclic heteroaromatic group having 1, 2 or 3 heteroatoms selected from O, N and S. The homo- and hetero-aromatic groups can be substituted with, for example, alkyl, halogen or nitro groups. Typical sulphonate leaving groups are mesylate (methanesulphonate), besylate (benzenesulphonate), tosylate (4-methylbenzenesulphonate), brosylate (4-bromobenzenesulphonate), nosylate (4-nitrobenzenesulphonate), triflate (trifluoromethanesulphonate), tresylate (2,2,2-trifluoroethanesulphonate) and 1-imidazolesulphonate groups.

Herein, the term “alkyl”, unless otherwise stated, preferably means a linear or branched chain saturated hydrocarbon group with 1-6 carbon atoms, such as methyl, ethyl, n-propyl, i-propyl, n-butyl, i-butyl, s-butyl, t-butyl or n-hexyl.

The term “C₃₋₈ cycloalkylidene” or “C₅₋₈ cycloalkylidene” preferably means a cycloalkylidene group optionally substituted with alkyl(s) wherein the cycloalkyl group with the optional substituent(s) is of 3-8 or 5-8 carbon atoms, respectively, such as cyclopropyl, cyclopentyl, cyclohexyl, cycloheptyl or 4,4-dimethyl-cyclohexyl. Particularly preferably, the cycloalkylidene group is a cyclopentylidene or cyclohexylidene group, and most preferably a cyclohexylidene group.

Herein, the term “base” preferably means an alkali metal or alkaline-earth metal hydroxide, alkoxide or carbonate, such as LiOH, NaOH, KOH, Mg(OH)₂, Ca(OH)₂, Ba(OH)₂, NaOMe, NaOEt, KO^{t}Bu, Li₂CO₃, Na₂CO₃, NaHCO₃, K₂CO₃ or BaCO₃. Strong basic ion exchange resins and tetraalkylammonium hydroxides are also suitable bases for use in this method. Preferably, the base is a hydroxide, alkoxide or carbonate, especially one of the following: LiOH, KOH, K₂CO₃, Ba(OH)₂ or particularly preferably Ca(OH)₂ or NaOH.

Herein, the term “reducing complex metal hydride” preferably means a salt wherein the anion contains a hydride moiety and therefore is capable of acting as a nucleophilic reducing agent by providing a hydride ion. In general, a complex metal hydride has the formula MₓM′_{y}Hₙ, where M is an alkali or alkaline-earth metal cation or a cation complex and M′ is a metal or metalloid, especially boron or aluminium. One or more hydride moieties can be replaced by an alkoxide, alkylamino, carboxylate, alkyl or cyano group. Typical examples of borohydrides and aluminium hydrides include LiBH₄, KBH₄, Ca(BH₄)₂, Zn(BH₄)₂, tetrabutylammonium borohydride, NaBH(OMe)₃, NaBH₃NMe₂, NaBH₃NH^{t}Bu, tetrabutylammonium triacetoxyborohydride, LiBHEt₃, lithium or potassium tris(sec-butyl)borohydride, KBHPh₃, sodium cyanoborohydride, tetrabutylammonium cyanoborohydride, LiAlH₄, NaAlH₄, KAlH₄, Mg(AlH₄)₂, LiAlH(OMe)₃, LiAlH(OEt)₃, LiAlH₂(OEt)₂, LiAlH(O^{t}Bu)₃, LiAlH(OCEt₃)₃ and NaAlH₂(OCH₂CH₂OMe)₂. Preferably, the complex metal hydride is a borohydride or an aluminium hydride, especially one of the following borohydrides: sodium, lithium, potassium, calcium and zinc borohydride, particularly preferably sodium borohydride.

The steps of the method of this invention—wherein a compound of formula 2 is treated with a reducing complex metal hydride and optionally a base—are simple and can be carried out simultaneously or in succession. The steps of this method can therefore be carried out either in one-pot or the intermediates formed in its steps can be isolated.

This method can be suitably carried out in any conventional aprotic solvent that does not contain functional group(s) susceptible to hydride attack (such as an ester, ketone or halogen group). Such solvents include ether type solvents such as diethyl ether, diisopropyl ether, THF and dioxane, and hydrocarbon solvents, preferably aromatic hydrocarbons such as benzene, toluene, xylene and mixtures thereof. When a borohydride is the reagent of choice, water or C₁₋₄ alcohols such as methanol, ethanol, isopropanol, or mixtures thereof also can be used as the solvent, preferably water or aqueous isopropanol.

When a base is used in the method, any conventional solvent can be used except for those that are susceptible to nucleophilic attack by a hydroxide or alkoxide. Typically, alkoxides can be added in C₁₋₄ alcohols at 20-100° C. Carbonates and hydroxides can be added in water, alcohol or water-organic solvent mixtures, in homogeneous or heterogeneous reaction conditions at temperatures varying from 0-100° C.

The reagents can be added together in one-pot reaction or sequentially, and the appropriate (common) reaction conditions for the reagents can be selected from those described above.

One way of carrying out this method is by treating the compound of formula 2 with only the reducing complex metal hydride to produce the compound of formula 1.

Another, preferred, way of carrying out this first method is by treating the compound of formula 2 simultaneously with the reducing complex metal hydride and the base to give the compound of formula 1.

Still another way of carrying out this method is by adding the reagents sequentially. Thus, in a first step a) a compound of formula 2 is treated with the reducing complex metal hydride to form a compound of formula 3

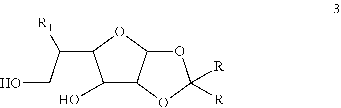

wherein R and R₁ are as defined above,
   and in a second step b), the compound of formula 3 is treated with the base and the reducing complex metal hydride to form the compound of formula 1. In the second step b), the reagents can be added simultaneously or sequentially. If the reagents are added sequentially, step b) comprises step b1) wherein the compound of formula 3 is treated with the base to form a compound of formula 4

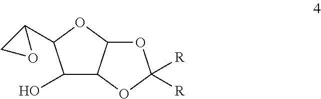

wherein R is as defined above,
   and step b2) wherein the resulting compound of formula 4 is treated with the reducing complex metal hydride to form the compound of formula 1.

A compound of formula 2 is preferably made by sulphonylating a compound of formula 5

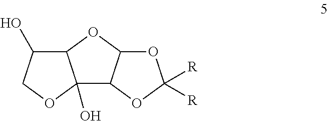

wherein R is as defined above.

Sulphonylating a compound of formula 5 to make a compound of formula 2 can be carried out in a conventional manner, preferably using a slight excess of a sulphonylating agent (˜1.5-3 equiv.), with or without added base, in an aprotic solvent such as toluene, THF, DCM, chloroform, dioxane, acetonitrile, chlorobenzene, ethylene dichloride, DMF, N-methylpyrrolidone, or mixtures thereof. The sulphonylating agent is preferably an activated sulphonyl derivative such as a halogenide or an anhydride, wherein the sulphonyl group is of the formula —SO₂—R*. Typical sulphonylating agents include mesyl chloride, besyl chloride, tosyl chloride, trifluoromethanesulphonic anhydride, etc. Tertiary amine bases such as pyridine, substituted pyridine (such as dimethylamino-pyridine), N,N-dimethylaniline, triethyl amine, Hünig's base, and the like are preferably added to the reaction mixture to scavenge acid by-products, particularly pyridine, substituted pyridine, N,N-dimethylaniline. Preferably, in the resulting sulphonylated compound of formula 2, R₁ is mesylate, besylate, tosylate, triflate, nosylate, brosylate or tresylate, particularly mesylate.

The compounds of formulae 1 to 5 contain several chiral carbon atoms, and therefore, each can exist as any of its diastereoisomers or as a mixture thereof. Preferably, the cyclic substituents on the tetrahydrofuran ring of each compound are in a relative cis-configuration. It also preferred that the compounds of formulae 1 to 5 are derived from D-glucose. Thus, it is preferred that the compound of formula 1 is in the form shown in formula 6,

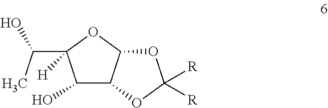

the compound of formula 2 is in the form shown in formula 7,

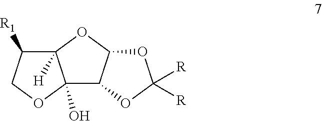

the compound of formula 3 is in the form shown in formula 8,

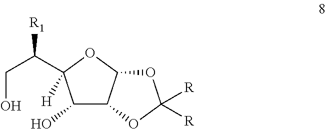

the compound of formula 4 is in the form shown in formula 9,

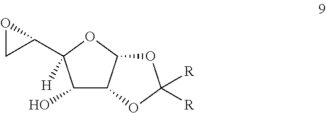

and the compound of formula 5 is in the form shown in formula 10,

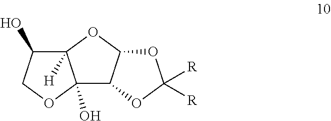

wherein R and R₁ are as defined above.

It is especially preferred that the optionally substituted 1,2-O-methylidene protecting group on each of the compounds of formulae 6-10 is isopropyl idene (R is methyl) or C₅₋₈ cycloalkylidene (the two geminal R-groups with the carbon atom to which they are attached form a C₅₋₈ cycloalkyl), particularly cyclopentylidene or cyclohexylidene, and most preferably cyclohexylidene.

In the process of this invention, a compound of formula 10 can be easily synthesized from D-glucose. See Scheme 2 below. In a first step a 1,2:5,6-di-O-alkylidene-α-D-glucofuranose derivative 11 can be formed formed by subjecting a keto derivative of formula R—CO—R, wherein R is as defined above (such as acetone, cyclohexanone, etc.) or a dialkyl acetal, preferably dimethyl acetal (e.g. 2,2-dimethoxy-propane) to acid catalysis. The 3-OH group of the compound of formula 11 can then be oxidized giving rise to the corresponding ulose derivative 12, wherein R is as defined above. A suitable oxidizing agent can be, e.g., a chromium(VI) reagent (CrO₃-pyridine complex, Jones reagent, PCC, pyridinium dichromate, trimethylsilyl chromate, etc.), MnO₂, RuO₄, CAN, TEMPO or DMSO in combination with one of DCC, Ac₂O, oxalyl chloride, tosyl chloride, bromine, chlorine, etc. The ulose derivative 12 can then be carefully treated with mild acid (typically 60-80% acetic acid) to deprotect the terminal glycol moiety selectively to give a keto-alcohol, which tends to spontaneously cyclize into a hemiacetal of formula 10.

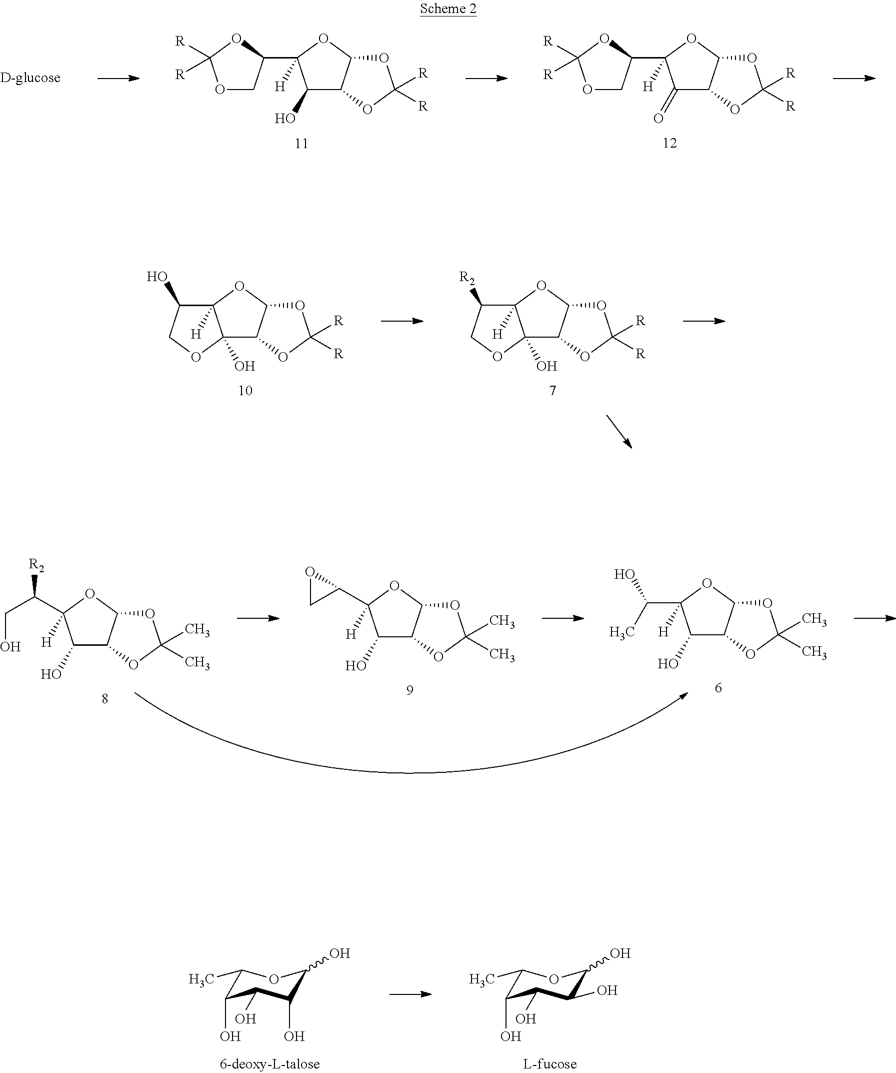

A compound of formula 6 can be readily converted into 6-deoxy-L-talose by acidic hydrolysis. Water, besides being the reagent, can serve as a solvent. Protic acids, such as acetic acid, trifluoroacetic acid, HCl, formic acid, sulphuric acid, perchloric acid, oxalic acid, p-toluenesulfonic acid, benzenesulfonic acid or cation exchange resins, can be used in amounts ranging from catalytic to a large excess.

Temperatures between 20° C. and reflux can be used for periods of 1 hour to 3 days, depending on temperature, concentration and pH. Preferably, HCl and organic acids, and particularly preferably aqueous solutions of acetic acid, formic acid, chloroacetic acid, oxalic acid, cation exchange resins, etc. are used at a temperature in the range of 40-90° C., preferably 40-75° C. (Zsoldos-Mády et al. Monatsh. Chem. 117, 1325 (1986).

Optionally, 6-deoxy-L-talose can be epimerized in the presence of molybdic acid to yield L-fucose (Defaye et al. Carbohydr. Res. 126, 165 (1984); Hricoviniova Tetrahedron: Asymmetry 20, 1239 (2009), WO 2011/144213).

The conversion of D-glucose into 6-deoxy-L-talose via the key intermediate of formula 7 of the process of this invention is depicted in Scheme 2 above.

By following Scheme 2,6-deoxy-L-talose can readily be made from 0-glucose with at least two steps fewer than previously required and with improved yields.

Also by following Scheme 2, the intermediates of formulae 6-12 can be isolated as crystalline materials. This is an important advantage since crystallization or recrystallization is one of the simplest and cheapest methods to: i) isolate a product from a reaction mixture, ii) separate it from contaminants and iii) obtain a pure product. Indeed, isolation or purification by crystallization generally makes any process more attractive and cost-effective industrially.

Certain intermediates of formulae 6-10—which are the compounds of formulae 13 and 14—

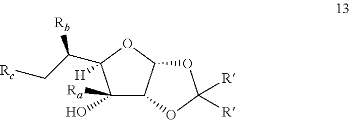

wherein the moiety

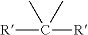

is a highly lipophilic protecting group and wherein either: Rₐ and R_{c} together form an oxygen bridge when R_{b} is OH or a sulphonate leaving group; or Rₐ is H and R_{c} is OH when R_{b} is a sulphonate leaving group,

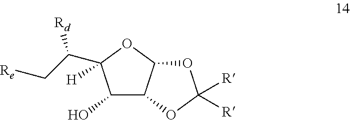

wherein the moiety

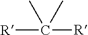

is a highly lipophilic protecting group, and wherein either: R_{d} is OH and Rₑ is H; or R_{d} and Rₑ together form an oxygen bridge,
   are the second aspect of this invention. The compounds of formulae 13 and 14 can be crystalline solids, oils, syrups, precipitated amorphous material or spray dried products. If crystalline, these compounds can be in either anhydrous or hydrated crystalline form by incorporating one or several molecules of water into their crystal structures. Similarly, these compounds can be crystalline substances incorporating ligands such as organic molecules and/or ions into their crystal structures.

Surprisingly, the steps of Scheme 2 provide relatively high yields of process intermediates of formulae 13 and 14. Their highly lipophilic ketal protecting groups make these intermediates more lipophilic and thus more soluble in organic solvents. This feature allows the use of smaller volumes of organic solvents and/or a smaller number of purification extractions, rendering the method steps even more efficient, quicker and more cost-effective, especially in large or industrial scale operations.

Additionally, the process intermediates of formulae 13 and 14 are preferably crystalline materials. Crystallization or recrystallization is one of the simplest and cheapest methods to isolate a product from a reaction mixture, separate it from contaminants and obtain the pure substance. Isolation or purification that uses crystallization makes the whole technological process robust and cost-effective, and thus advantageous and attractive compared to other procedures. However, the compounds of formulae 13 and 14 can also be in the form of oils, syrups, precipitated amorphous material or spray dried products. The preferred compounds of formulae 13 and 14 are those of formula 15

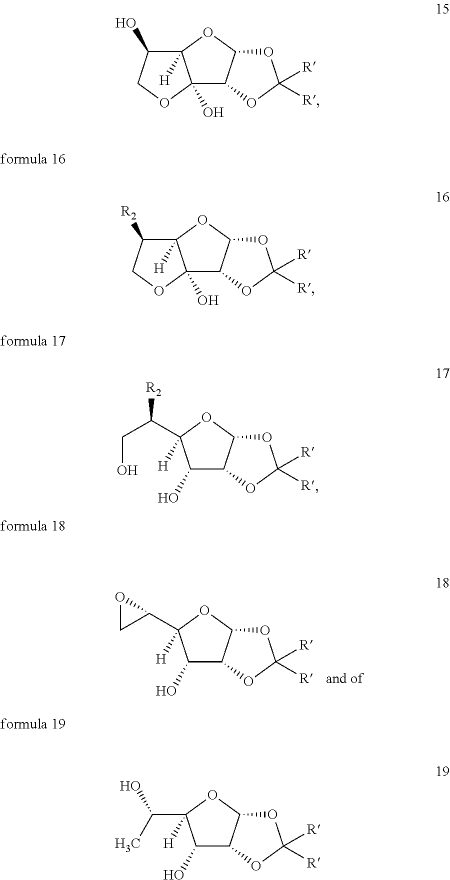

in which the two geminal R′-groups together with the carbon atom to which they are attached form a cycloalkylidene group, preferably a cyclohexylidene group, and thereby are crystalline. Particularly preferred are the compounds of formulae 16 and 17 in which R_{b} is mesylate, besylate, tosylate, triflate, nosylate, brosylate or tresylate, and particularly preferred are those in which R_{b} is mesylate or tosylate.

Other features of the invention will become apparent in view of the following exemplary embodiments which are illustrative but not limiting of the invention.

### EXAMPLES

### Example 1

### Compounds of Formula 10

To a solution of sodium bicarbonate (0.06-0.07 equiv.) in water (200 mL), acetone (200-250 mL), ruthenium dioxide hydrate (0.02 equiv.), sodium bromate (0.45-0.55 equiv.) and 1,2:5,6-di-O-alkylidene-α-D-glucofuranose (a compound of formula 11, 360-390 mmol) were added portionwise. The reaction mixture was stirred for 3-8 h at room temperature (22° C.; “rt”), then isopropanol (0.4-0.5 equiv.) was added, and the mixture was stirred for further 2-4 h. After filtrating the solid residue, HCl-solution (0.10-0.25 equiv.) was then added to the filtrate, and the resulting mixture was kept at 25-55° C. for 2-5 h under continuous stirring. NaOH or NaHCO₃ (1.0-1.1 equiv. to HCl) in water was added to the reaction mixture which was extracted with ethyl acetate (100-200 mL) after 30 min. The phases were separated, the aqueous phase was extracted with ethyl acetate (100-200 mL), the combined organic phases were evaporated and the resulting syrupy residue was crystallized.

R=methyl, yield 90%

¹H NMR (CDCl₃, 300 MHz): δ=5.97 (d, 1H, H-1), 4.48 (m, 1H, H-5), 4.44-4.42 (m, 2H, H-2, H-4), 4.24 (m, 1H, H-6a), 3.78 (m, 2H, H-6b, OH-3), 2.53 (s, 1H, OH-5), 1.59 (d, 3H, CH₃), 1.39 (d, 3H, CH₃). M.p.: 80-81° C.

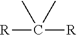

=cyclohexylidene, yield: 84%

¹H NMR (CDCl₃, 300 MHz): δ=5.98 (d, 1H, H-1), 4.48 (m, 1H, H-5), 4.40 (m, 2H, H-2, H-4), 4.22 (m, 1H, H-6a), 3.83-375 (m, 2H, OH-3, H-6b), 2.58 (m, 1H, OH-3), 1.82-1.36 (m, 10H, CH₂ cyclohexylidene). M.p.: 108-110° C.

### Example 2

### Compounds of Formula 7

Sulphonyl chloride (1.1 eq.) was slowly added to a mixture of a compound of formula 10 (2.0 g) and pyridine (4 mL) at 0° C. The mixture was allowed to warm to rt under stirring or heated to 50° C. After completion of the reaction (1-24 h), the reaction mixture was cooled to 0° C., water (1 mL) was added followed by HCl-solution (2 mL) and ethyl acetate (10 mL). The phases were separated, the aqueous phase was extracted with ethyl acetate (10 mL), and the combined organic phases were washed with saturated sodium bicarbonate (5 mL) and brine (5 mL). The organic phase was evaporated to dryness to afford an oily syrup which was crystallized or purified by column chromatography.

R=methyl, R₁=mesyloxy, yield: 66%

¹H NMR (CDCl₃, 300 MHz): δ=5.98 (d, 1H, H-1), 5.23 (m, 1H, H-5), 4.59 (m, 1H, H-2), 4.42-4.37 (m, 2H, H-4, H-6a), 4.02 (m, 1H, H-6b), 3.70 (s, 1H, OH-3), 3.08 (s, 3H, CH₃ mesyl), 1.49 (d, 3H, CH₃), 1.36 (d, 3H, CH₃). M.p.: 115-117° C.

R=methyl, R₁=tosyloxy, yield: 65%

¹H NMR (CDCl₃, 300 MHz): δ=7.81 (d, 2H, tosyl), 7.19 (d, 2H, tosyl), 5.93 (d, 1H, H-1), 5.01 (m, 1H, H-5), 4.38-4.21 (m, 3H, H-2, H-4, H-6a), 3.91 (m, 1H, H-6b), 3.70 (s, 1H, OH-3), 2.42 (s, 3H, CH₃ tosyl), 1.45 (d, 3H, CH₃), 1.28 (d, 3H, CH₃). M.p.: 80-81° C.

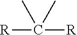

=cyclohexylidene, R₁=mesyloxy, yield: 68%

¹H NMR (CDCl₃, 300 MHz): δ=6.01 (d, 1H, H-1), 5.22 (m, 1H, H-5), 4.59 (m, 1H, H-2), 4.43-4.38 (m, 2H, H-4, H-6a), 4.03 (m, 1H, H-6b), 3.79 (s, 1H, OH-3), 3.06 (s, 3H, CH₃ mesyl), 1.79-1.27 (m, 10H, CH₂ cyclohexylidene). M.p.: 135-137° C.

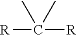

=cyclohexylidene, R₁=tosyloxy, yield: 41%

¹H NMR (CDCl₃, 300 MHz): δ=7.79 (d, 2H, tosyl), 7.15 (d, 2H, tosyl), 5.88 (d, 1H, H-1), 4.88 (m, 1H, H-5), 4.14-4.08 (m, 3H, H-2, H-4, H-6a), 3.89 (m, 1H, H-6b), 3.60 (s, 1H, OH-3), 2.39 (s, 3H, CH₃ tosyl), 1.63-1.15 (m, 10H, CH₂ cyclohexylidene). Syrup.

### Example 3

### Compounds of Formula 6 (One-Pot Procedure)

A: Sodium borohydride (15 equiv.) was added to a solution of a compound of formula 7 (0.17 mmol) in isopropanol (2 mL) and water (0.4 mL) at rt. After stirring 24 h at rt, the reaction mixture was evaporated to dryness, and a) the residue was purified by column chromatography to afford pure compound which was optionally crystallized (R=methyl), or b) the residue was partitioned between DCM and water, and after separation the DCM was evaporated and the product was crystallized (R—C—R=cyclohexylidene).

R=methyl, yield: 58%

¹H NMR (CDCl₃, 300 MHz): δ=5.78 (d, 1H, H-1), 4.48 (m, 1H, H-2), 3.87-3.78 (m, 2H, H-3, H-5), 3.53 (m, 1H, H-4), 1.50 (d, 3H, CH₃), 1.31 (d, 3H, CH₃), 1.24 (d, 3H, H-6). M.p.: 92-94° C.

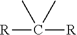

=cyclohexylidene, yield: 86%

¹H NMR (CDCl₃, 300 MHz): δ=5.80 (d, 1H, H-1), 4.54 (m, 1H, H-2), 3.89-3.81 (m, 2H, H-3, H-5), 3.58 (° C. m, 1H, H-4), 1.78-1.32 (m, 10H, CH₂ cyclohexylidene), 1.22 (d, 3H, H-6). M.p.: 68-70° C.

B: Calcium hydroxide (1.2 equiv) and sodium borohydride (1.3 equiv.) were added to a solution of a compound of formula 7 (6.7 mmol) in water (6 mL) at 50° C., and the mixture was stirred for 3 h. The resulting suspension was filtered, and the filtrate was evaporated to dryness. The residue a) was purified by column chromatography to afford pure compound which was optionally crystallized (R=methyl), or b) was partitioned between DCM and water, and after separation the DCM was evaporated and the product was crystallized

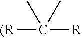

=cyclohexylidene). Spectroscopic data were identical with those obtained in procedure A.

The reaction was also carried out replacing calcium hydroxide with Na₂CO₃, NaHCO₃, NaOH and K₂CO₃.

R=methyl, yield: 70-78%

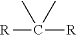

=cyclohexylidene, yield: 75-81%

### Example 4

### Compounds of Formula 6 Via Compound of Formula 8

Sodium borohydride (0.3 equiv.) was added to a solution of a compound of formula 7 (6.7 mmol) in water (6 mL) at 0° C., and the mixture was stirred for 0.5 h. TLC showed consumption of starting material and formation of a new compound which proved to be a compound of formula 8.

R=methyl and R₁=mesyloxy: ¹H NMR (CDCl₃, 300 MHz): δ=5.98 (d, 1H, H-1), 4.85 (m, 1H, H-5), 4.62 (m, 1H, H-2), 4.20 (m, 1H, H-3), 4.08-3.82 (m, 3H, H-4, H-6a, H-6b), 3.70 (s, 1H, OH-3), 3.30 (s, 1H, OH-6), 3.08 (s, 3H, CH₃ mesyl), 1.59 (d, 3H, CH₃), 1.26 (d, 3H, CH₃).

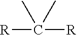

=cyclohexylidene and R₁=mesyloxy: ¹H NMR (CDCl₃, 300 MHz): 8=5.80 (d, 1H, H-1), 4.90 (m, 1H, H-5), 4.61 (m, 1H, H-2), 4.18 (m, 1H, H-3), 4.06-3.84 (m, 3H, H-4, H-6a, H-6b), 3.50 (s, 1H, OH-3), 3.18 (s, 1H, OH-6), 3.08 (s, 3H, CH₃ mesyl), 1.82-1.35 (m, 10H, CH₂, cyclohexylidene).

To the resulting mixture, calcium hydroxide (1.2 equiv) and sodium borohydride (1.0 equiv.) were added at 50° C. and the mixture was stirred for 3 h. The suspension was filtered and the filtrate was evaporated to dryness. The residue a) was purified by column chromatography to afford pure compound which was optionally crystallized (R=methyl), or b) was partitioned between DCM and water, and after separation, the DCM was evaporated and the product was crystallized (R—C—R=cyclohexylidene). Spectroscopic data were identical with those obtained in Example 3.

The reaction was also carried out replacing calcium hydroxide with Na₂CO₃, NaHCO₃, NaOH and K₂CO₃.

R=methyl, yield: 80-88%

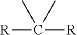

=cyclohexylidene, yield: 69%

### Example 5

### Compounds of Formula 6 Via Compounds of Formulae 8 and 9

Sodium borohydride (0.3 equiv.) was added to a solution of a compound of formula 7 (6.7 mmol) in water (6 mL) at 0° C., and the mixture was stirred for 0.5 h. TLC showed consumption of starting material and formation of a new compound which proved to be a compound of formula 8. To the resulting mixture, calcium hydroxide (1.2 equiv) was added at rt. After 15 min, TLC showed consumption of compound of formula 8 and formation of a new compound of formula 9.

R=methyl: ¹H NMR (CDCl₃, 300 MHz): δ=5.98 (d, 1H, H-1), 4.58 (m, 1H, H-2), 3.89 (m, 1H, H-3), 3.63 (m, 1H, H-4), 3.17 (m, 1H, H-5), 2.82 (m, 2H, H-6), 2.50 (s, 1H, OH-3), 1.56 (d, 3H, CH₃), 1.36 (d, 3H, CH₃). M.p.: 62-64° C.

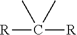

=cyclohexylidene: ¹H NMR (CDCl₃, 300 MHz): δ=5.88 (d, 1H, H-1), 4.50 (m, 1H, H-2), 3.91 (m, 1H, H-3), 3.61 (m, 1H, H-4), 3.13 (m, 1H, H-5), 2.79 (m, 2H, H-6), 2.42 (s, 1H, OH-3), 1.78-1.31 (m, 10H, CH₂ cyclohexylidene).

Then sodium borohydride (1.0 equiv.) was added at 50° C., and the mixture was stirred for 3 h. The suspension was filtered, and the filtrate was evaporated to dryness. The residue a) was purified by column chromatography to afford pure compound which was optionally crystallized (R=methyl), or b) was partitioned between DCM and water, and after separation the DCM was evaporated and the product was crystallized (R—C—R=cyclohexylidene). Spectroscopic data were identical with those obtained in Example 3.

The reaction was also carried out replacing calcium hydroxide with Na₂CO₃, NaHCO₃, NaOH and K₂CO₃.

R=methyl, yield: 80-88

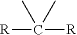

=cyclohexylidene, yield: 69%

### Example 6

### Partition Studies

1,2-O-Cyclohexylidene-6-deoxy-β-L-talofuranose (197 mg) was partitioned between water (10 mL) and methylene chloride (10 mL), layers separated and the residual amount of solutions in the separatory funnel were partitioned with extra amount of water (5 mL) and methylene chloride (5 mL). The combined organic phases were evaporated and dried in vacuo (50° C., <1 mbar, 1 hour) to give 140 mg of the title compound. The aqueous solution gave 53 mg after lyophilisation and drying in vacuo (50° C., <1 mbar, 2 hrs). Using the same procedure, partition between ethyl acetate and water provided 127 mg of the title compound from ethyl acetate phase and 70 mg from the aqueous phase.

Analogously, 1,2-O-isopropylidene-6-deoxy-β-L-talofuranose (150 mg) was partitioned between methylene chloride (24 mg) and aqueous phase (126 mg). Partition between ethyl acetate and water furnished 7 mg and 143 mg of the title compound, respectively. The results are summarized in the following table:

| | | | | Number of |
|---|---|---|---|---|
| | Solvent | Partition P | | volumes of organic |
| Compound | system | (=[C]_{org}/[C]_{aq}) | logP | solvent* (yield) |
| 1,2-O-cyclohexylidene-6- | CH₂Cl₂-water | 2.64 | 0.42 | 3 (98%) |
| deoxy-β-L-talofuranose | | | | |
| 1,2-O-cyclohexylidene-6- | ethyl acetate- | 1.81 | 0.26 | 3 (95.5%) |
| deoxy-β-L-talofuranose | water | | | |
| 1,2-O-isopropylidene-6- | CH₂Cl₂-water | 0.19 | 0.72 | 18 (95.6%) |
| deoxy-β-L-talofuranose | | | | |
| 1,2-O-isopropylidene-6- | ethyl acetate- | 0.16 | 0.79 | 21 (95.6%) |
| deoxy-β-L-talofuranose | water | | | |

*To reach at least 95% of recovery using one volume of organic solvent each time relative to one volume of aqueous solution.

These results show that the highly lipophilic cyclohexylidene group on compound 6, which is a protected 6-deoxy-L-talose derivative, resulted in compound 6 having a higher affinity to organic solvents as compared to aqueous media. This implies a surprisingly much higher solubility of compound 6 in organic solvents as compared to aqueous solvents, which tremendously facilitates its extraction into an organic solvent. By comparison, the corresponding isopropylidene compound had higher affinity to aqueous medium, therefore was highly soluble in aqueous solutions and almost insoluble in organic solvents.

1. A method of making a compound of formula 1 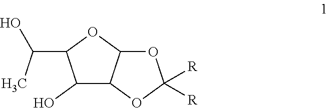
wherein R is independently H, alkyl or phenyl or wherein the two germinal R groups together with the carbon atom to which they are attached form a C₃₋₈ cycloalkylidene group,
comprising the step of:
treating a compound of formula 2 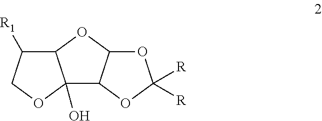
wherein R is as defined above and R₁ is a sulphonate leaving group,
with a reducing complex metal hydride to form the compound of formula 1.

2. The method according to claim 1, wherein the compound of formula 2 is treated simultaneously with the reducing complex metal hydride and a base.

3. The method according to claim 1, comprising the steps of:
a) treating a compound of formula 2 with the reducing complex metal hydride to form a compound of formula 3 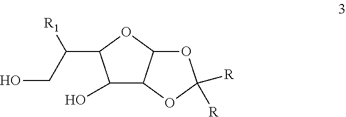
wherein R and R₁ are as defined above, and
b) treating the compound of formula 3 with a base and the reducing complex metal hydride to form the compound of formula 1.

4. The method according to claim 3, wherein step b) comprises the steps of:
b1) treating the compound of formula 3 with the base to form a compound of formula 4 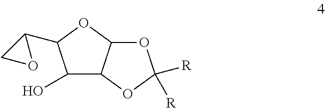
wherein R is as defined above, and
b2) treating the compound of general formula 4 with the reducing complex metal hydride to form the compound of formula 1.

5. The method according to claim 2, wherein the base is selected from the group consisting of alkali metal and alkaline-earth metal hydroxides, alkoxides and carbonates, and the reducing complex metal hydride is selected from the group consisting of borohydrides and aluminium hydrides.

6. The method according to claim 5, wherein the alkali metal and alkaline-earth metal hydroxide is selected from LiOH, NaOH, KOH, Ba(OH)₂ and Ca(OH)₂, and and the borohydride is selected from sodium, lithium, potassium, calcium and zinc borohydride.

7. The method according to claim 1, wherein the compound of formula 2 is prepared by sulphonylating a compound of general formula 5 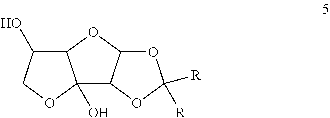
wherein R is as defined above.

8. The method according to claim 1, wherein R₁ is selected from the group consisting of mesylate, besylate, tosylate, triflate, nosylate, brosylate and tresylate.

9. The method according to claim 1, wherein the compound of formula 1 is in the form shown in formula 6, 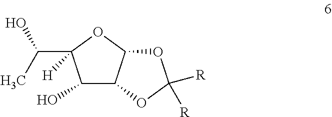
and the compound of formula 2 is in the form shown in formula 7, 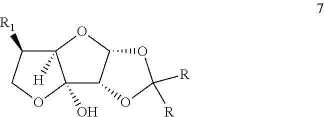
wherein R and R₁ are as defined above.

10. The method according to claim 9, wherein the compound of formula 7 is prepared by sulphonylating a compound of formula 10 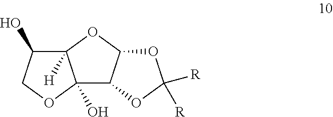
wherein R is as defined above.

11. The method according to claim 1, wherein R is independently a highly lipophilic C₂₋₆ alkyl or phenyl group, or wherein the two R groups together with the carbon atom to which they are attached form a highly lipophilic C₅₋₈ cycloalkylidene group.

12. The method according to claim 11, wherein the two geminal R-groups together with the carbon atom to which they are attached form a cyclohexylidene group.

13. (canceled)

14. The method according to claim 9, wherein a compound of formula 6 is treated with an acid to form 6-deoxy-L-talose, which is optionally converted into L-fucose by epimerization.

15. A process for making L-fucose from D-glucose comprising the method according to claim 1.

16. A compound of formula 13 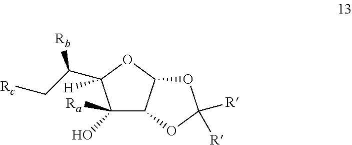
wherein the moiety 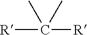
is a highly lipophilic protecting group and wherein either: Rₐ and Rₑ together form an oxygen bridge when R_{b} is OH or a sulphonate leaving group; or Rₐ is H and Rₑ is OH when R_{b} is a sulphonate group,
or formula 14 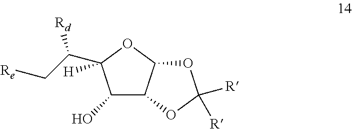
wherein the moiety 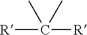
is a highly lipophilic protecting group, and wherein either: R_{d} is OH and Rₑ is or R_{d} and Rₑ together form an oxygen bridge.

17. The compound according to claim 16, wherein the moiety 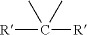
is a hydrocarbon group of at least 5 carbon atoms.

18. The compound according to claim 17, wherein, in the moiety 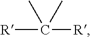
R′ is a C₂₋₆ alkyl or phenyl group, or wherein the two geminal R′ groups together with the carbon atom to which they are attached form a C₅₋₈ cycloalkylidene group.

19. The compound according to claim 18, wherein, in the moiety 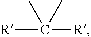
the two geminal R′ groups together with the carbon atom to which they are attached form a cyclohexylidene group.

20. The compound according to claim 16 that is isolated in crystalline form.

21. (canceled)

22. The method according to claim 1, wherein the two geminal R groups together with the carbon atom to which they are attached form a C₃₋₈ cycloalkylidene group.

23. The method according to claim 3, wherein the base is selected from the group consisting of alkali metal and alkaline-earth metal hydroxides, alkoxides and carbonates, and the reducing complex metal hydride is selected from the group consisting of borohydrides and aluminium hydrides.

24. The method according to claim 4, wherein the base is selected from the group consisting of alkali metal and alkaline-earth metal hydroxides, alkoxides and carbonates, and the reducing complex metal hydride is selected from the group consisting of borohydrides and aluminium hydrides.

25. The method according to claim 23, wherein the alkali metal and alkaline-earth metal hydroxide is selected from LiOH, NaOH, KOH, Ba(OH)₂ and Ca(OH)₂, and the borohydride is selected from sodium, lithium, potassium, calcium and zinc borohydride.

26. The method according to claim 24, wherein the alkali metal and alkaline-earth metal hydroxide is selected from LiOH, NaOH, KOH, Ba(OH)₂ and Ca(OH)₂, and the borohydride is selected from sodium, lithium, potassium, calcium and zinc borohydride.

27. The method according to claim 6, wherein the alkaline-earth metal hydroxide is Ca(OH)₂ and the borohydride is sodium borohydride.

28. The method according to claim 25, wherein the alkaline-earth metal hydroxide is Ca(OH)₂ and the borohydride is sodium borohydride.

29. The method according to claim 26, wherein the alkaline-earth metal hydroxide is Ca(OH)₂ and the borohydride is sodium borohydride.

30. The method according to claim 8, wherein R₁ is mesylate or tosylate.

31. The method according to claim 3, wherein the compound of formula 3 is in the form shown in formula 8, 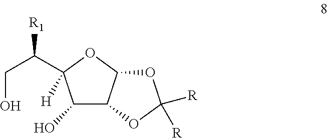
wherein R and R₁ are as defined above.

32. The method according to claim 4, wherein the compound of formula 4 is in the form shown in formula 9, [chemical structure]
